(19) United States
(12) Patent Application Publication
(10) Pub. No.: US 2003/0170180 A1
(43) Pub. Date: Sep. 11, 2003

(54) Non-lethal chemical weapons

(76) Inventor: William S. Bahary, Pearl River, NY (US)

Correspondence Address: DR. WILLIAM S. BAHARY, 291 N. MIDDLETOWN RD, PEARL RIVER, NY, 10965, US

(21) Appl. No.: 10/293,527

(22) Filed: Nov. 12, 2002

Related U.S. Application Data
(60) Provisional application No. 60/353,312, filed on Nov. 13, 2001.

(51) Int. Cl. A61K 49/00; A61K 9/68

(52) U.S. Cl. 424/10.4

(57) ABSTRACT

The invention relates to compositions of non-lethal chemical weapons for riot and crowd control. The compositions can also be used to clear areas and facilities for extended periods without collateral damage. Specifically, this invention discloses “dial an effect” non-lethal weapons for crowd control, from moderate to near lethal strength. The composition contains three unique active ingredients comprising an odoriferous substance, a hot substance, and a tear gas agent, A method is disclosed to prepare the compositions with solvents, carriers, pressurizers, and optional ingredients, as well as a method to apply them.

### FIELD OF THE INVENTION

The invention relates to non-lethal weapons. Specifically, the purpose of the invention is to disclose compositions and methods of using the compositions for crowd control. Further, the objective is to ward off enemies and specifically terrorists by making them move out of locations in proximity to civilians, and of their hiding places. The compositions range from the non-lethal to near lethal ones, with the non-lethal compositions sufficiently objectionable to make the whereabout places of objectionable subjects uninhabitable.

### BACKGROUND

In the 21^{st} century, combat with terrorists is of prime concern. Because of the terrorists elusive and destructive towards the military as well as civilians, the availability of an array of non-lethal weapons is an important option in combination with lethal weapons. Terrorists are necrophiles and appear to be instinctively compelled to death and destruction.

Many countries and nations experience serious problems with terrorists. For example, in Afghanistan, the terrorists can hide in caves and tunnels to avoid detection. In the 18^{th} century, the British could not conquer the Afghani's. More recently, in the 20^{th} century, the Russians could not conquer Afghanistan. After many years of fighting, the Russians were forced to withdraw from their positions in Afghanistan.

The problems facing the Russian armies were enormous. For example, the terrorist would hide in the mountains along the main highways. As the Russian armies were traveling in convoys along the narrow highways, the terrorists would shoot from high in the mountains and disable the first and last vehicles in the caravan, and cause havoc. Another problem in fighting the terrorists is that they would hide in the face of the oncoming army. After the army would establish themselves, the terrorists would come out of their hiding places and snipe at the Russian position, so that the Russian army would have to re-conquer and re-establish themselves at high material and human costs.

Bombing the terrorists in their hiding places has its shortcomings. Some of the hiding places in the caves are deep in rocky mountains, so that many of the caves remain intact after extensive bombing. Both the terrorists and their equipment may remain undamaged.

One approach in controlling crowds and terrorists is to use chemical warfare. Chemicals in war were reviewed, for example, by B. Harris, in Kirk Othmer's Encyclopedia of Chemical Technology, Vol. 5, 795-816, 4^{th} Ed. 1998. These include the toxic agents, examples of which are the lethal mustard gases and nerve agents. These chemicals pose some ethical questions. They also pose practical issues, since other terrorists can return to the hiding places after a relatively short period, as the chemical agents loose their potency. Other chemical agents include flames, incendiaries, and smoke, but these also do not render the residence places uninhabitable.

Another approach is to use non-lethal weapons. These may be irritants, aversives, repellents, or mal-odrants that would make crowds leave an area, especially if the enemy is intermingling with civilians. The non-lethal weapons of the prior art for crowd control, include tear gas and pepper spray. However, it is believed that the compositions and methods of this invention are significantly superior to those of the prior art. The use of non-lethal weapons was recently described in a book entitled: An Assessment of Non-Lethal Weapons Science and Technology (2002)”, published by the National Academies Press.

### PRIOR ART

The primary chemical agents used in crowd control are tear gas and pepper sprays. The following overview of the prior art of riot control agents is not meant to be exhaustive, but representative of the substances involved. This review will show the superiority of the compositions of this invention over the prior art for crowd control.

Tear Gas. Tear gases is the common name for substances which, in low concentrations, cause pain in the eyes, flow of tears and difficulty in keeping the eyes open. Apart from the effect on the eyes, most tear gases cause irritation in the nose and mouth, throat and airways, and sometimes also in the skin. The strongly irritating effect leads to a pronounced incapacitation of the exposed persons. These can lead to nausea and vomiting.

Among a long series of more than 15 substances used worldwide as tear gas agents, three have become more prominent. These substances are alpha-chloroaceto-phenone (codename CN), dibenzoxazepine (codename CR) and ortho-chlorobenzylidene-melanonitrile (codename CS). Currently, CS is the most widely used tear gas.

At room temperature, these tear gases are white solid substances. They are stable when heated so that they are usually dispersed as an aerosol by heating or by dissolving in an organic solvent. CN and CR are stable in water and difficult to decompose under practical conditions, whereas agent CS is rapidly hydrolyzed in water and inactivated.

The threshold concentration for an intolerable effect (TC-50) and the incapacitating concentrations (IC-50) of these compounds are low, whereas the lethal doses (LD50) are high. For example, for compound CS, the above values are 0.004, 4.0, and 100,000 mg/m³, respectively. That is the acute toxicity of these tear gases is low, as the margin between the concentration giving an intolerable effect and that which may cause injury or death is high.

The effects of agent CS are immediate, even in extremely low concentrations. The median concentration for eye effects is 1-5 mg/m³ or about 1-5 parts per million. The onset of maximum effects is 20-60 s. The effect is reversible since the duration of the effect is 5-10 min after the individual has been removed to fresh air.

Although CS was used in the Vietnam war in a continuous spray in cave and tunnel systems, its effectiveness is limited because the compound is a solid and will not permeate corners like a gas. A further difficulty with these tear gases is that they are chemically not stable with time in the atmosphere.

Allyl isothiocyanate is another lacrimator or tear gas agent, but this has a very low melting point and is a gas at room temperatures. However, allyl isothiocyanate is more toxic and more reactive, so that there may be a problem with stability.

In a more complete review, Zarc International company literature at Zarc.com describes lung irritants, vesicants or blistering agents, irritant smokes and sternutators (sneezing), lacrimators and nerve agents. In addition to the above tear-gas agents, some others include alpha bromobenzyl cyanide, dibromodimethyl ether, dichlorodimethyl ether, ethyl dibromoarsine, and phenylcarbylamine chloride. The three prominent nerve gas agents are phosphine compounds and may be used as a last resort. No mention is made of the compounds of this invention.

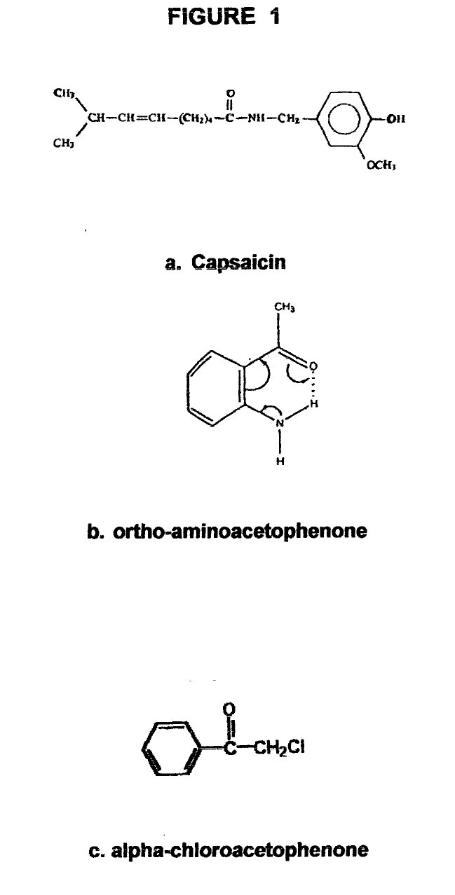

Capsaicin and peppers. During the past two decades, capsaicin and pepper sprays (codename OC) have become popular with law enforcement and corrections personnel as non-lethal deterrent agents. The principle active ingredient in OC sprays used by police and others as antipersonnel agents is oleoresin capsicum (OC), an oily extract of pepper plants of the genus Capsicum. OC extract consists of a complex mixture of fat-soluble phenols known as capsaicinoids. Capsaicin (trans-8-methyl-N-vanyllyl-6-nonenamide) and dihydrocapsaicin are the most potent homologues and make up 80 to 90% of the total. See FIG. 1a. The capsaicionoid content determines the hotness of the extract. The relative hotness is measured in Scoville units, the greatest dilution of pepper extract that can be detected by the human tongue. The capsaicinoid content of the extracts used in pepper sprays varies from 1.2% to about 12%, and the amount of the extract in pepper sprays varies, so that the potential risks associated with capsaicinoid exposure may vary as much as 30-fold among brands of OC spray. (C. Smith et. al., J. Applied Physiol. 1985, vol. 58, 1080-4).

Depending on the brand, an OC spray may contain water, alcohol, or organic solvents as carriers, and nitrogen, carbon dioxide, or halogenated hydrocarbons (such as tetrachloroethylene and methylene chloride) as propellants to discharge the canister contents.

The health effects of capsaicin and OC include dermal, respiratory, nasal and ocular irritation. Dermal exposure to OC causes redness, swelling and intense burning pain. Respiratory and nasal responses include coughing, sneezing and inability to breathe or speak. Eye symptoms include redness, severe burning pain, and involuntary closing of the eyelids. The tear gas agents CN and CS produce primarily irritant effects, but exposure to OC causes both irritation and neurogenic inflammation. The effect of OC is also more immediate than that of tear gas. Although the effects of OC spray disappear after a couple of hours, serous adverse effects, even death, have followed the use of OC sprays. Commercially, Cap-stun^{R} from Zarc International contains about 0.92% capsaicinoid, and enables users to incapacitate nearly all subjects.

The difficulty with the “hot” pepper sprays for use against terrorists is that they are most effective on contact and the product does not disseminate itself.

Calmatives have recently been used, but are out of the scope of this invention.

Other Repellents and Aversives. Among a long list of chemical substances used as animal repellents, the following are most noteworthy.

Mason, Clark, and Shah (J. Wildlife Manage, 55 (2), 334-340, 1991), evaluated ortho-aminoacetophenone (OAP), along with three other isomers, para-amino-acetophenone, meta-aminoacetophenone, and alpha-aminoacetophenone with respect to repellency to birds in a one or two cup water feeding test and found the ortho-aminoacetophenone to be the most effective. They further showed that OAP is about ten times more repellent than methyl anthranilate (MA). That is, whereas MA may be used at 1% concentration, OAP may require 0.1% to yield the same repellency.

Nolte, Campbell and Mason tested the repellent efficacy of five compounds with respect to water intake by mice. Among the five compounds: OAP, MA, aminomethoxy-acetophenone, 2-methoxyacetophenone, and veratryl amine, OAP was found to be the most aversive with respect to water intake by mice.

U.S. Pat. No. 5,464,625 (1995), by Nolte, Mason and Clark discloses numerous substances for repellency to rodents that would prevent them from consuming crops and livestock feeds. The 19 compounds tested with respect to water consumption by mice included ortho-, meta- and para-aminoacetophenones; ortho-, meta-, and para-methoxyacetophenones; ortho-, meta-, and para-hydroxyaceto-phenones, methyl anthranilate, veratryl amine, acetophenone, 2-aminobenzyl alcohol, 2-amino-4,5-dimethoxyacetophenone, anthranilamide, anthranilic acid, isatoic anhydride, isobutyl anthranilate, 4-ketobenztriazine, methoxybenzoic acid, methyl cinnamate, methyl salicylate, phenethyl anthranilate, and sodium benzoate. The most repellent material was OAP at 1.0% and 0.5% concentrations. At 0.5% concentration, all the compounds exhibited some repellency except isatoic anhydride, methyl cinnamate, methyl salicylate, phenyl anthranilate, and sodium benzoate. In the above patent the rodents mentioned were squirrels, rats, mice, voles, mountain beavers, pocket gophers, and hares. The materials noted for protection were crops seeds, seedlings, telephone cables, electric cables and refuse containers. Humans and crowd control were not mentioned.

U.S. Pat. No. 5,849,364 by Nachtman and Hull discloses a self foaming sprayable composition consisting of water, surfactant, clay mineral, water soluble polymer and a glue to cover waste materials as in a landfill to prevent the emission of unpleasant odors. The composition can optionally contain a pest control agent to repel birds, animals and insects so they don't spread disease or become a nuisance. The bird aversion agents included alpha-aminoacetophenones, ortho-hydroxyaceto-phenone, ortho-, meta-, and para-methoxyacetophenone, methyl anthranilate, and dimethyl anthranilate. The most preferred compound was methyl anthranilate.

U.S. Pat. No. 5,672,352 (1997) by Clark, Mason, Shah, et. al. discloses methods for identifying the avian repellent effects of a compound using drinking assays, and methods of repelling birds from materials susceptible to consumption by birds. A long list of compounds was evaluated using their drinking assays and field tests. The compounds included most of the ones mentioned in U.S. Pat. No. 5,464,625 which includes the ortho-, meta-, and para-derivatives of aminoacetophenone, hydroxyacetophenone, and methoxyacetophenone, MA, and dimethyl anthranylate. They reported that OAP was more repellent to birds than the isomers of methoxy- or hydroxy-acetophenones, and MA.

U.S. Pat. No. 6,024,971 (2000) by Nachtman and Hull discloses a method for repelling birds which uses a water fog. The method consists of forming a mixture of water, methyl anthranilate and yucca or a surfactant as a dispersing agent. The composition is sprayed from a container under pressure to form a repelling fog. Although several bird repelling compounds including various isomers of amino-acetophenone, hydroxyacetophenone, and methoxyacetophenone are mentioned, MA is preferred.

Other aversives include odoriferous substances that result from protein degradation, which include blood meals, rotted egg formulations and fish products. Avoidance is mediated in these cases by sulfur compounds and volatile acids, for example. These substances can repel by olfaction as well as gastrointestinal malaise. Deer repellent products based on degraded proteins and fish are currently on the market. Other repellents include semiochemicals, examples of which are chemicals in urine or glandular products, e.g. the skunk odor butyl mercaptan. There is evidence that they may cause animals and humans to leave area. Semiochemicals and products from protein degradation are difficult to use in large quantities with an army and may pose environmental and health problems.

Still other non-lethal weapons include visual, acoustic, electrical shock and laser techniques, but these techniques may not be practical in remote mountainous areas or for dealing with terrorists intermingled with civilians.

Further, Mason in an overview of mammal repellents, teaches that there are marked differences in sensitivity and/or perception towards irritants between different taxonomic groups (Proc. 18^{th} Vertebr. Pest Conf., Baker & Crabb,eds. Published at Univ. of Calif., Davis, 1998). That is, substances that irritate mammals rarely affect birds and vice versa. For example, although, capsaicin is universally repellent to mammals at concentrations between 10 to 100 PPM, birds are indifferent to capsaicin concentrations of upwards of 20,000 PPM. On the other hand, methyl anthranilate repels birds at concentrations well below those that are offensive to most mammals.

None of these patents or references mentioned OAP for use with humans. Further, the combinations of OAP with OC and/or tear gas was not mentioned, which are the primary objectives of this invention.

### SUMMARY OF INVENTION

It is the object of this invention to disclose a composition to employ as a non-lethal weapon for crowd control. It is also to be used as an effective way to displace terrorists from their hideouts and make their hideouts uninhabitable with minimum damage to the environment and minimum harm to civilians.

The primary composition of this invention is a malodorant. OAP is present in some variety of grapes and has a pleasant odor at low concentrations. However, it has been unexpectedly discovered that at relatively moderate concentrations or with extended exposure, OAP is a repulsive malodorant to humans. It has further been unexpectedly discovered that it is a strong permeant and disperses very rapidly beyond what might be expected from its molecular weight. It is believed that it can be a useful and practical non-lethal weapon for crowd control and warfare in urban centers.

In a first embodiment of this invention, a non-lethal irritating odoriferous substance is used to control crowds. It further can make physical locations, examples of which are houses and caves uninhabitable, even to terrorists. The preferred odoriferous substance of this invention may be ortho-aminoacetophenone, although some others may be used as described below.

In a second and more repellent embodiment of this invention, a malodorant substance is used in combination with a tear gas agent, or in combination with a capsaicinoid hot substance.

In a third and most repellent embodiment of this invention, an odoriferous substance is used in combination with a tear gas agent and a capsaicinoid hot substance. The combination of aversives of this invention is superior to each of the individual aversives. Furthermore, there exists an unexpected synergy in using a combination of these aversives so that the combined effect is greater than the arithmetic sum of the effect of the individual aversives. The odoriferous substance can be mixed with an effective carrier and dispersed under pressure from a container by way of application, either alone or in combination with the other mentioned aversives.

### DETAILS OF THE INVENTION

1.Odoriferous Substance. The first component of the formulation of this invention is an odoriferous substance that the targeted individuals may detect immediately. These affect the olfactory senses and add a major dimension to the composition. Examples of odoriferous compounds useful here include OAP. Examples of other odoriferous compounds that have aversive affects are derivatives of anthranilic acid, including but not limited to meta- or para-aminoacetophenone, or methyl anthranylate. The odor of OAP is noticeable in a few parts per billion level and smells like lambrusca grapes. However, it was unexpectedly found to be irritating at higher concentrations. Over time, it becomes increasing repulsive.

The ortho-aminoacetophenone structure is shown in FIG. 1b, and its molecular weight is 135 daltons. It is a solid at room temperature and at low PPM concentrations it has the pleasant odor of lambrusca grapes. However, at higher concentrations OAP has a very strong and offensive odor, the irritation of which does not diminish with time. It is believed that its aversiveness is due both to its olfactory irritation and general incapacitating effect so that the affected person requires fresh air. Any repulsively odoriferous compound may be used and may include the following compounds: ortho-aminoacetophenone, meta-aminoacetophenone, or para-aminoacetophenone, ortho-hydroxyacetophenone meta-hydroxyacetophenone, or para-hydroxyacetophenone, ortho-methoxyacetophenone, meta-methoxyacetophenone, or para-methoxyacetophenone, methyl anthranilate (MA), dimethy anthranilate, veratryl amine, aminomethoxyacetophenone, other ring compounds, and other highly odoriferous substances. Ortho-aminoacetophenone is preferred. OAP has a high vapor pressure to yield a high concentration in air. It permeates and disperses rapidly in air to go around corners and get into crevices. Since its odor is detectable in parts per billion, and it permeates and disperses rapidly in air, it is especially effective as a non-lethal weapon.

Although the concept of using a combination of aversive stimuli is familiar, there is little evidence that multiple stimuli are more effective than the individual components (Avery and Mason, Crop Protection, Vol. 16, No. 2, pp. 159-164, 1997). The identification of the most appropriate combination of stimuli for a given species will require laboratory and field testing to determine the degree of efficacy and efficiency. Efficacy and efficiency refer to the amount of material needed to provide the desired effect and efficiency refers to the length of time the material is effective. Additivity or synergy in a composition is unobvious and can not be assumed.

It is believed that the formulation contemplated herein used by itself or containing the following additional aversive ingredients is a novel and effective composition to use as a non-lethal weapon. This composition permeates and disseminates itself better than those of the previous art. The composition of this invention lasts longer than those of the previous art, and may last upwards of a year, depending on the amount of material sprayed, and the composition employed. Further, if the composition is sprayed on ceilings, the material permeates and will be more difficult to cover or to eliminate the irritating odor, than if sprayed on the floor.

2. Hotness agent The second component of the formulation of this invention is a hot substance. For hotness, several compounds may be employed containing capsaicin, the principal hot substance found in hot peppers, including cayenne peppers, habanero peppers and oleoresin capsicum (OC). The oleoresin capsicum extract consists of a complex mixture of fat-soluble phenols known as capsaicinoids. Capsaicin (trans-8-methyl-N-vanillyl-6-nonenamide) and dihydrocapsaicin are the most potent homologues, and make up 80 to 90% of the total. Capsaicinoid content determines the hotness of the extract and the capsaicinoid content of the OC extract may range from 1.2% to 12.6%. Natural or synthetic capsaicin may also be used and diluted as required. Other derivatives of vanillylamine that are hot may be used. The preferred compounds are the capsaicin and the oleoresin capsicum. Capsaicin is considered to be a strong irritant to humans.

2. Tear Gas. Tear gases and lacrimators are compounds that cause copious flow of tears. These are irritants and cause other physiological effects that include: coughing, difficulty in breathing, chest tightness, involuntary closing of the eyes, runny nose and dizziness. Heavy concentrations cause vomiting and nausea.

Substances that can be used as tear gas in this invention include the following.

1. Lung irritants: diphenylcyanoarsine (DC), fluorine, and trichloronitro methane (PS).

2. Vesicants or blister agents: B-chlorovinyldichloroarsine (L), bis(dichloroethyl)-sulfide (mustard gas, HD), ethyldichloroarsine (ED), methyldichloroarsine.

3. Irritant smoke and sternutators (sneezing): diphenylamine chloroarsine (DM), diphenyochloroarsine (BA).

4. Lacrimators and tear-gases: dibenzoxazepine (CR), chloroacetophenone (CN), orthochlorobenzalmalononitrile (CS), alpha bromobenzyl cyanide (BBC), dibromo-dimethyl ether, dichlorodimethyl ether, ethyl dibromoarsine, and phenylcarbylamine chloride and allyl isothiocyanate.

The preferred compounds of this invention are compound CN and CS. Although CS is a solid at room temperature, it is disseminated as a spray, as a cloud of dust, or as an aerosol. When dusted on the ground, CS may remain active for as long as five (5) days. When used with a silicone water repellent, the activity may persist as long as 45 days.

4. Other ingredients. Other components contemplated herein may be a carrier or carriers, which may include water, a dispersing agent, an organic solvent, and a polymer as described below. The system also may include a propelling agent, examples of which are nitrogen, carbon dioxide or freon. The carriers may be as solids, liquids, or gases.

The invention described herein comprises the following embodiments. These embodiments allow for the rheostat effect or dial an effect to produce mild non-lethal weapons to near lethal weapons on demand.

In a first embodiment of this invention, an irritating malodor substance is used to control crowds and to clear areas or facilities. The odoriferous substance may include any of the following: ortho-aminoacetophenone, meta-aminoacetophenone, or para-aminoacetophenone, ortho-hydroxyacetophenone, meta-hydroxyacetophenone, or para-hydroxyacetophenone, ortho-methoxyacetophenone, meta-methoxyacetophenone, or para-methoxyacetophenone, methyl anthranilate, dimethy anthranilate, veratryl amine, and other highly odoriferous substances. Ortho-aminoacetophenone is preferred. Its odor is noticeable in parts per billion, but its non-oral toxic dose (TDLo) is 1500 mg/kg.

In a second and more impeding embodiment of this invention, an odoriferous substance is used in combination with a tear gas agent, or in combination with a capsaicinoid hot substance. The tear gas agent may include compound CS or alpha-chloroacetophenone (CN). The structure of CN is shown in FIG. 1c.

In a third and most dissuasive or repellent embodiment of this invention, an odoriferous substance is used in combination with a tear gas agent and a capsaicinoid hot substance. The combination of aversives of this invention is superior to each of the individual aversives. Furthermore, there exists an unexpected synergy in using a combination of these aversives so that the combined effect is greater than the arithmetic sum of the effect of the individual aversives.

It is believed that the “hotness” of OC and lacrimating of tear gas stimulate different receptors so that the combination elicits an enhanced repelling response. As for the odoriferous substances, OAP has a strong repulsive odor at parts per million concentrations and has a bad taste, so that the combination of a third type of receptor is involved to enhance efficacy. Additionally, the utilization of a combination of materials enhances the population of a given species deterred and broadens the spectrum of people repelled. It is further believed that the malodor will last upwards of a year depending on the amount of material used and the carrier/composition as described below.

### METHODS

In the first embodiment, the odoriferous substance may be dissolved in a suitable solvent and sprayed under pressure from a container with a nozzle. Suitable solvents These aversives may be employed in several different methods and processes. include alcohols, and methylene chloride, and other organic solvent, with isopropyl being preferred. Pressurizing substances include nitrogen, carbon dioxide and freon. Alternatively, the odoriferous substance can be dispersed in water using a dispersant examples of which include cationic, anionic or nonionic or other surfactants, yucca, or lecithin, or mixtures thereof. The dispersion or emulsion can be dispensed as above under pressure. In the preferred first embodiment, OAP is dissolved in isopropyl alcohol and dispensed from a spray gun under nitrogen pressure.

In the second embodiment, in which the odoriferous substance is used in combination with capsaicinoids or tear gas, the substances are added to the organic solvent, since all are organic soluble and dispensed as above,. Alternatively, the capsaicinoid or tear gas can be dispersed in water using a dispersant and combined with the dispersion of the odoriferous substance. These mixtures can be dispensed under pressure as above.

In the third embodiment, all three aversives may be dissolved in an organic solvent an example of which is isopropyl alcohol, and dispensed as above. Alternatively, the capsaicinoid and tear gas can be dispersed in water using a dispersant and combined with the dispersion of the odoriferous substance. This mixture can be dispensed under pressure as above from a container.

In another approach, one or more of the aversives are dissolved in a small amount of organic solvent. Then the solution is added to an emulsions of low to medium molecular weight surface-active agent in water with stirring to disperse or emulsify the ingredients of the composition. Some examples of surface-active agents that fit this category are anionic, cationic, nonionic or amphoteric surfactants, examples of which are sodium laurate or sodium lauryl sulfate or sufonates, in processes well known to anyone familiar with the art of surfactants, emulsifiers or dispersants.

A higher molecular weight surface-active agent also known as polymeric surfactants or emulsifiers as well as film forming polymers or silicones may be optionally added. On application and drying, the polymers leave a film on the aversives. This makes the active aversives longer lasting than if no polymer was used, as the polymeric film makes the actives evaporate or dissipate slower. The polymer may be an acrylate polymer or copolymer, starch, natural gum, or a polysilicone.

Among the methods used to dispense the compositions of this invention are riot shotguns, fog generators, grenades or bombs. A common modern weapon is the handheld aerosol tear-gas projector known as Mace. Still other methods of application of the solutions or emulsions are to dispense them from a pressurized container or to use a spray gun. U.S. Pat. No. 4,895,273 describes a semi automatic device for applying tear gas.

The proportions of the aversives in the blend can be 0.01 to 99.9% of tear-gas, 0.01 to 99.9% of OC, and 0.01 to 98% of OAP. A preferred range is 0.1-90% tear-gas, 0.1-90% OC, and 0.01-60% OAP. A more preferred range is 1-80% tear-gas gas, 1-60% OC, and 0.1-40% OAP. Still, a most preferred proportion is 10-60% OC, 10-60% tear gas, and 10-60% OAP.

The concentration of the blend of active aversives in the composition may be 2-90% or the maximum that can be dissolved at moderate temperatures. A more preferred range is 10-70% and a most preferred range of the total actives concentration depends on location and size and feasibility. In a commando attack, the individual will want to carry the lightest load so that the more concentrated composition will be preferred.

It is believed, and the examples suggest that malodorant materials including OAP are effective non-lethal substances to displace crowds and clear areas. One example is to clear terrorists from their hiding places. Combinations comprising OAP with OC or OAP with tear-gas or combinations of all three types of aversives are synergistically superior people repellents. Each component elicits a response from different receptors so that the combination is superior to any single part. The composition of this formulation not only affects a larger population, but because of their synergistic effect, the composition of this invention is more repulsive. The composition disperses effectively and is long lasting. Over time it is nauseating and disorienting.

### EXAMPLES OF ANIMAL TESTS

The following examples illustrate the application of the aversives of this invention and in no way are meant to limit its scope. In the following examples I-X, naive squirrels were employed as test animals. These were individually placed in a metal screen cage 12×12×24 inches long, with a special food compartment blocked by the placement of four 6×¼ inch removable wooden dowels. These squirrels were fed seeds and grains for pets and whole peanuts in shells.

### Examples I-IV

In these examples, squirrel 1 was acclimated to the cage for several days with unlimited access to food and water. Then the food compartment was blocked with the four dowels, and the time it took the squirrel to break the dowels to access the food was recorded. In experiments I-III, the dowels were untreated and it took the squirrel about 2-4 hours to break the dowels. In example IV, the dowel was treated with OAP by applying some OAP to the six inch dowel with a Q-tip. About 0.1-0.2 GMs was added to a 4 g dowel. In this case, it took the squirrel 22 hours to access the food as summarized in Table I. This confirms the observation that OAP is a repellent to rodents. The fact that the rodents did finally eat is consistent with the notion that rodents will not starve themselves because their food is unpalatable or smelly, and that survival instinct is dominant.

### Examples V-VII

In these three examples, the second naive squirrel (2) was acclimated and then exposed to the treated OAP dowels first after acclimation. In the absence of any choice for food, it took 25 hours before the squirrel broke the dowels to access the food, Example V. However, if there was a choice for another food, an apple as in Example VI, the time passed before the squirrel broke the dowels to access the food increased to over 28 hours, as shown in Table II. This again confirms the deterrent effect of OAP. When untreated dowels were placed to block the food with this squirrel, the time passed to access the food was still 26 hours, instead of the previous 2-4 (Example VII). This suggests conditioning of the squirrel to avoid the dowels, which means that humans also will continue to be repulsed by areas where the compositions have been sprayed.

### Examples VIII-X (CAPSAICIN)

In these experiments, a third naive squirrel (3) was tested with Capsaicin treated dowels after acclimation as usual. In Example VII, it took the squirrel 28 hours to break the dowels in the absence of any other choice for food. If there was a choice of another food, an apple, the access time increased to 31 hours (Example IX). Further, in the control Example X where the dowel was untreated and there was a choice for an apple, the access time was 42 hours as shown in Table III. This is again consistent with the notion that rodents will not starve because the Capsaicin is unpalatable and repelling. Example X suggests that after conditioning, the squirrel will still avoid the dowels as expected. In conclusion, these experiments demonstrate that both OAP and CAP are repellents to squirrels and by extension repulsive to humans.

### Example of Human Tests

A few grams of OAP were placed in a glass jar with a tight cap. The cap was further sealed with duct tape, and the jar was further wrapped in aluminum foil and sealed in two plastic bags. It was unexpectedly found that the odor was noticeable after a few days, months, and even after one year. The odor was especially repulsive on the warmer summer days, when it permeated the enclosed space as well as the surrounding outdoor space.

Further, although the odor of OAP was initially pleasant at very low concentration in the order of parts per billion or less, at slightly higher concentrations the odor became unexpectedly repulsive, dissuading, and impeding. Still further, over time the odor at low concentration became irritating and impeding, as it elicited a sensitized reaction.

TABLE I
EFFECT OF OAP TREATMiENT ON TIME TO
BREAK DOWELS NO CHOICE

| | TREATMENT- | TIME TO BREAK | |
|---|---|---|---|
| ITEM | TYPE | DOWELS | CHOICE |
| EXAMPLE I | NONE-CONTROL 1 | 4 | NO |
| EXAMPLE II | NONE-CONTROL II | 1.5 | NO |
| EXAMPLE III | NONE-CONTROL III | 2.5 | NO |
| EXAMPLE IV | OAP | 22 | NO |

TABLE II
EFFECT OF OAP TREATMENT ON TIME TO BREAK
DOWELS OR 2 CHOICES

| | TREATMENT | TIME TO BREAK | |
|---|---|---|---|
| ITEM | TYPE | DOWELS | CHOICE |
| EXAMPLE V | OAP | 25 HOURS | NO |
| EXAMPLE VI | OAP | OVER 28 | YES |
| EXAMPLE VII | OAP | 26 | YES |

TABLE III
EFFECT OF CAPSAICIN TREATMENT ON TIME TO
BREAK DOWELS 1 OR 2 CHOICES

| | TREATMENT | TIME TO BREAK | |
|---|---|---|---|
| ITEM | TYPE | DOWELS | CHOICE |
| EXAMPLE VIII | CAPSAICIN | 28 HOURS | NO |
| EXAMPLE IX | CAPSAICIN | 31 | YES |
| EXAMPLE X | NONE | 42 | YES |

I claim:

1. A non-lethal composition containing a malodorant substance, that can effectively control crowds and that can be used to clear areas and facilities for extended periods.
1a. A composition of claim 1 wherein the malodorant is OAP.

2. A stronger non-lethal composition consisting of a malodorant substance of claim 1 in combination with a hotness substance or a tear gas agent, that can effectively control crowds and that can be used to clear areas and facilities for extended periods.
2a. A composition of claim 2 wherein the malodorant is OAP, the hot substance is OC, and the tear gas is CS.

3. A near lethal composition containing a malodorant substance of claim 1, in combination with a hotness substance, and a tear gas agent, that can effectively control crowds and that can be used to clear areas and facilities for extended periods. It can further make the areas/facilities uninhabitable for extended periods.
3a. A composition of claim 3 wherein the malodorant is OAP, the hot substance is OC, and the tear gas is CS.

4. A composition wherein the odoriferous substance is ortho-aminoacetophenone, the hotness agent is oleoresin capsicum and the tear gas agent is alpha-chloroacetophenone, compound CN, or compound CS. The composition may contain a carrier, pressurizing agent, dispersing agent, and a polymeric substance.

5. A method for applying the compositions of claim 1-4 to control crowds or to clear areas and facilities in a practical manner, comprising the following steps:
a. Dissolve the active agents in a suitable solvent.
b. Apply the dissolved actives from a pressurized container.
